(19) United States
(12) Patent Application Publication
(10) Pub. No.: US 2009/0318556 A1
(43) Pub. Date: Dec. 24, 2009

(54) BIOMARKERS FOR DETECTING RADIATION EXPOSURE: METHODS AND USES THEREOF

(76) Inventors: Jeffrey R. Idle, Roztoky (CZ); Frank J. Gonzalez, Bethesda, MD (US); John B. Tyburski, Gaithersburg, MD (US); Kristopher W. Krausz, Bethesda, MD (US); Andrew David Patterson, Montgomery Village, MD (US)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 12/121,208

(22) Filed: May 15, 2008

(51) Int. Cl. A61K 31/195; C07C 63/06; C07C 229/02; C07H 19/06; C07C 309/03; C07D 473/04; C07H 19/16; G01N 33/48; G01N 33/68; C12Q 1/02; G01N 33/00; A61P 43/00; G01N 27/447

(52) U.S. Cl. 514/562; 562/426; 562/553; 536/28.53; 562/104; 544/267; 536/27.21; 536/28.5; 436/98; 436/94; 436/120; 436/89; 435/29; 422/68.1; 536/28.4; 204/461

(57) ABSTRACT

Disclosed herein are biomarkers for determining gamma radiation exposure by an animal or human. The biomarkers include 3-hydroxy-2-methylbenzoic acid 3-O-sulfate, N-hexanoylglycine, β-thymidine, taurine, xanthine, xanthosine, 2′-deoxyuridine, 2′-deoxycytidine, 2′-deoxyxanthosine, or any salt, ion, or combination thereof. Also disclosed are methods for determining gamma radiation exposure by an animal or human, which include the step of measuring the amount of one or more biomarkers specific to gamma radiation in the biological fluid and correlating the amount of said biomarkers to the amount of gamma radiation exposure by the animal or human. Systems for determining gamma radiation exposure by an animal or human and methods of treating an animal or human for gamma radiation exposure are also disclosed.

### STATEMENT OF GOVERNMENT INTEREST

The invention was made with U.S. Government support. The Government may have certain rights in the invention under Grant U19 AI067773-02 from the National Institute of Allergy and Infectious Diseases and by the National Cancer Institute intramural program

### FIELD OF THE INVENTION

The present invention is in the field of metabolomics, also known as metabonomics or metabolic profiling. The present invention is also in the field of biomarkers. The present invention is also in the field of detecting radiation exposure in an animal or human, accordingly the present invention is also in the field of radiation dosimetry. The present invention is also in the field of systems for detecting radiation exposure as well as for treating animals and humans who have been exposed to gamma radiation.

### BACKGROUND OF THE INVENTION

Humans are exposed to ionizing radiation from several sources. Natural background radiation from galactic and solar cosmic rays, and terrestrial radionuclides of radon, potassium, uranium, and thorium represents about 80% of the average radiation exposure to Americans of 3.6 mSv per annum. The remaining exposure can be attributed to man-made radiation sources, including diagnostic X-rays, nuclear medicine and radiotherapy. Finally, various consumer products are a source of radiation exposures and these include televisions, watches, carbon-based fuels, smoke detectors, and fluorescent lamp starters. However, with the growing need for nuclear waste disposal into the environment and the ever-increasing threat of a terrorist nuclear event, it is now necessary to develop biomarkers of ionizing radiation exposure that can be used for mass screening in the event of a radiological mass casualty incident. Currently, there are no non-invasive means for radiation exposure assessment in research animals or humans. Accordingly, the identification of dosimetry biomarkers is a priority effort to prepare for a possible terrorist attack with radiological or nuclear devices, or in the event of a nuclear accident. To achieve these ends, high-throughput devices that evaluate radiological dose exposures need to be designed, developed, manufactured and employed to guide triage and subsequent therapeutic choices.

### SUMMARY OF THE INVENTION

Gamma radiation exposure has both short-term and long-term adverse health effects including cancer. The threat of modern terrorism places human populations at risk for radiological exposures, yet current medical countermeasures to radiation are limited. Metabolomics for γ (“gamma”) radiation biodosimetry in animals were determined. Mice were γ irradiated at doses of 0, 3, and 8 Gy (2.57 Gy/min), and urine samples collected over the first 24 h post-exposure were analyzed by ultra-performance liquid chromatography-time of flight mass spectrometry (UPLC-TOFMS). Multivariate data were analyzed by orthogonal partial least squares (OPLS) and using the random forests machine learning algorithm. Both 3 and 8 Gy exposures yielded distinct urine metabolomic phenotypes. The top cohort of ions for 3 and 8 Gy were further analyzed, including tandem mass spectrometric comparison with authentic standards, revealing that 2′-deoxyxanthosine, xanthosine, 2′-deoxyuridine, 2′-deoxycytidine, N-hexanoylglycine and β-thymidine are urinary biomarkers of 3 and 8 Gy exposure, 3-hydroxy-2-methylbenzoic acid 3-O-sulfate and xanthine are elevated in urine of mice exposed to 3 but not 8 Gy, and taurine is elevated after 8 but not 3 Gy exposure. Gene Expression Dynamics Inspector (GEDI) self-organizing maps showed clear dose-response relationships for subsets of the urine metabolome. Accordingly, the present invention is useful for identifying animals and humans exposed to γ radiation. The present invention is also useful for metabolomic strategies for noninvasive radiation biodosimetry in humans.

Accordingly, in one aspect of the present invention there are provided biomarkers for determining gamma radiation exposure by an animal or human, comprising a pyrimidine base, a purine base, a xanthine base, a pyrimidine nucleoside, a purine nucleoside, a xanthine nucleoside, a metabolite of a nucleic acid, a metabolite of fatty acid metabolism, or any salt, ion, or combination thereof.

In other aspects of the present invention are provided methods for determining gamma radiation exposure by an animal or human, comprising: (a) collecting a biological fluid from the animal or human; (b) measuring the amount of one or more biomarkers specific to gamma radiation in the biological fluid; and (c) correlating the amount of said biomarkers to the amount of gamma radiation exposure by the animal or human.

The present invention also provides systems for determining gamma radiation exposure by an animal or human, comprising: a sample introduction section for collecting a fluid sample; a volatilization section for volatilizing the fluid sample; an ion source for ionizing a portion of the volatilized sample; an ion mobility based filter for focusing at least one biomarker comprising an ion of a pyrimidine base, a purine base, a xanthine base, a pyrimidine nucleoside, a purine nucleoside, a xanthine nucleoside, a metabolite of a nucleic acid, a metabolite of fatty acid metabolism, or any combination thereof, and a detector for detecting the at least one biomarker.

Within additional aspects, the present invention provides methods for treating an animal or human exposed to gamma radiation, comprising: (a) collecting a biological fluid from the animal or human; (b) measuring the amount of one or more biomarkers specific to gamma radiation in the biological fluid to develop a radiation exposure profile of the animal or human; (c) correlating the radiation exposure profile to one or more compounds capable of counteracting the metabolic effects of the gamma radiation on the animal or human; and (d) administering the one or more compounds to the animal or human.

The general description and the following detailed description are exemplary and explanatory only and are not restrictive of the invention, as defined in the appended claims. Other aspects of the present invention will be apparent to those skilled in the art in view of the drawings, detailed description, and claims, as provided herein.

### BRIEF DESCRIPTION OF THE DRAWINGS

The summary, as well as the following detailed description, is further understood when read in conjunction with the appended drawings. For the purpose of illustrating the invention, there are shown in the drawings exemplary embodiments of the invention; however, the invention is not limited to the specific methods, compositions, and devices disclosed. In addition, the drawings are not necessarily drawn to scale. In the drawings:

FIG. 1. Twenty-four hour mouse urine sample collection protocol for radiation metabolomics. Two groups of mice were used for studies to identify γ radiation-specific changes in urine metabolites, one group destined for exposure (A, hv), and one for sham control handling (B, Sham). Urine collections were made in alternating periods whereby the mice spent 24 h individually in metabolic cages (—) and then 24 h in their holding room cages ( . . . ) with littermates. Three pre-exposure collections were taken to allow the animals to adapt to the handling and new environments. Urine was then collected for 24 h immediately following radiation and sham exposures.

FIG. 2. OPLS Scores and loadings plots for urine samples from control and irradiated mice. Component 1 (abscissa) scores for urine collected over the first 24 h post-exposure from control (O) and irradiated () mice using 3 Gy (B) and 8 Gy (A) doses show class separation based on exposure status. Ions are found to be elevated in urine from mice exposed to 3 Gy (C) and 8 Gy (D) compared to the respective controls. Loadings reveal ions in a spatial relationship to the class separation. At each dose, a cohort of ions was selected to serve as candidate biomarkers of radiation exposure, as indicated.

FIG. 3. Determination of the chemical structure of the 3 Gy biomarker #1 by tandem mass spectrometry. Top panel shows the negative ion MS/MS fragmentation of synthetic 3-hydroxy-2-methylbenzoic acid 3-O-sulfate, which eluted at 2.1 min. Bottom panel shows the negative ion MS/MS fragmentation of a peak in mouse urine, also eluting at 2.1 min.

FIG. 4. Fold increases in urinary creatinine and biomarkers at 3 and 8 Gy over sham irradiated animals. A. Creatinine; B. 3-Hydroxy-2-methylbenzoic acid 3-O-sulfate; C. N-Hexanoylglycine; D. β-Thymidine; E. Taurine; F. 2′-Deoxycytidine; G. Xanthine; H. Xanthosine; I. 2′-Deoxyuridine; J. 2′-Deoxyxanthosine. All data for the quantitated biomarkers (panels C-E) were normalized to creatinine concentration (μmol/mmol creatinine, see text). Data for 3-hydroxy-2-methylbenzoic acid 3-O-sulfate (panel B), 2′-deoxycytidine (panel F), xanthine (panel G), xanthosine (panel H), 2′-deoxyuridine (panel I) and 2′-deoxyxanthosine (panel J) are based upon relative peak areas and then normalized to creatinine (see text). Asterisks indicate statistically significant elevations over controls (see text). Data shown are means with 95% confidence intervals.

FIG. 5. Dose-response of the mouse urinary metabolome to γ radiation. A. Self-organizing maps that give a holistic view of the urinary metabolome in a 13×11 matrix (average of 42 ions per cell), constructed using GEDI software. Data used comprise approximately 6,000 negative ions (ESI− mode). B. Sum total of relative intensities of negative ions in a 3×3 matrix in bottom left-hand corner of the maps, with background subtraction (11 Gy values). There is a clear radiation dose-response decline in biomarkers in this region of the metabolome. C. Sum total of relative intensities of negative ions in a 3×3 matrix in bottom right-hand corner of the maps, with background subtraction (0 Gy values). There is a clear radiation dose-response increase in biomarkers in this region of the metabolome. D. Self-organizing maps that give a holistic view of the urinary metabolome in a 13×11 matrix (average of 42 ions per cell), constructed using GEDI software. Data used comprise approximately 6,000 positive ions (ESI+ mode). Note that the areas of the positive ion maps that increase and decrease with radiation dose correspond to the areas of the negative ion maps that increase and decrease with radiation dose (Panel A).

FIG. 6. Predictive ability of biomarker combinations for ionizing radiation exposure in mice. A. N-Hexanoylglycine and taurine (0 vs. 3 Gy). B. N-Hexanoylglycine and taurine (0 vs. 8 Gy). C. N-Hexanoylglycine and β-thymidine (0 vs. 3 Gy). D. N-Hexanoylglycine and taurine (0 vs. 8 Gy). E. Taurine and β-thymidine (0 vs. 3 Gy). F. Taurine and β-thymidine (0 vs. 8 Gy).

### DETAILED DESCRIPTION OF ILLUSTRATIVE EMBODIMENTS

The present invention may be understood more readily by reference to the following detailed description taken in connection with the accompanying figures and examples, which form a part of this disclosure. It is to be understood that this invention is not limited to the specific devices, methods, applications, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

It is to be appreciated that certain features of the invention which are, for clarity, described herein in the context of separate embodiments, may also be provided in combination in a single embodiment. Conversely, various features of the invention that are, for brevity, described in the context of a single embodiment, may also be provided separately or in any subcombination. Further, reference to values stated in ranges include each and every value within that range.

As used herein, the term “biomarker” means a substance whose detection indicates a particular disease state, for example, the presence of an antibody may indicate an infection. More specifically, a biomarker indicates a change in expression or state of a protein that correlates with the risk or progression of a disease, or with the susceptibility of the disease to a given treatment. A biomarker can be used to diagnose disease risk, presence of disease in an individual, or to tailor treatments for the disease in an individual through choices of drug treatment or administration regimes. In evaluating potential drug therapies, a biomarker may be used as a surrogate for a natural endpoint such as survival or irreversible morbidity. If a treatment alters the biomarker, which has a direct connection to improved health, the biomarker serves as a surrogate endpoint for evaluating clinical benefit. Accordingly, a biomarker can also be used to indicate exposure to various environmental substances or radiation, such as gamma radiation.

The biomarkers of the present invention for determining gamma radiation exposure by an animal or human, can include a pyrimidine base, a purine base, a xanthine base, a pyrimidine nucleoside, a purine nucleoside, a xanthine nucleoside, a metabolite of a nucleic acid, a metabolite of fatty acid metabolism. Various forms of these biomarkers are also suitable for determining gamma radiation exposure by an animal or human, such as a salt of the biomarker or an ion of the biomarker. Biomarker ions are denoted for their ability to be detected using any of a variety of ion mass spectrometry methods, the details of which are further described herein below. Any combination of the biomarkers, their salts, or their ions can also be used for determining gamma radiation exposure by an animal or human, as further described herein below.

Suitable pyrimidine nucleoside biomarkers include a pyrimidine 2′-deoxyriboside, a pyrimidine riboside. More specifically, suitable pyrimidine 2′-deoxyriboside biomarkers include β-thymidine, 2′-deoxyuridine, 2′deoxycytidine, or any salt, ion, or combination thereof. Suitable purine base biomarkers include xanthine, or any salt, ion, or combination thereof. Suitable purine nucleoside biomarkers can include a purine riboside, a purine deoxyriboside, or any salt, ion, or combination thereof. More specifically, suitable purine riboside biomarkers include xanthosine, 2′-deoxyxanthosine, or any salt, ion, or combination thereof.

The biomarkers of the present invention for determining gamma radiation exposure by an animal or human, may also be a metabolite of a nucleic acid, or any salt, ion, or combination thereof. Suitable metabolites of a nucleic acid that can be used as biomarkers herein include 3-hydroxy-2-methylbenzoic acid 3-O-sulfate, N-hexanoylglycine, β-thymidine, taurine, xanthine, xanthosine, 2′-deoxyuridine, 2′-deoxycytidine, or any salt, ion, or combination thereof. The biomarkers of the present invention may be a metabolite originating in one or more cells of the animal or human, or they may be a metabolite originating in the flora contained within the gut of the animal or human. Accordingly, the biomarkers include metabolites of a nucleic acid is characterized as a metabolite of a flora contained within the gut of the animal or human. Specifically, a suitable metabolite of the flora contained within the gut of the animal or human for use as a biomarker according to the present invention includes 3-hydroxy-2-methylbenzoic acid 3-O-sulfate. Suitable metabolites of a nucleic acid for use as a biomarker herein can also be characterized as a marker for liver damage, such as taurine.

The biomarkers of the present invention for determining gamma radiation exposure by an animal or human, may also be a metabolite of fatty acid metabolism, or any salt, ion, or combination thereof. Suitable metabolites of fatty acid metabolism can originate in the cells of the liver as well as in cells in other organs. A suitable biomarker that is a metabolite of fatty acid metabolism found in the liver includes N-hexanoylglycine. Any ion or salt of N-hexanoylglycine can also be used as a biomarker, including any combination with N-hexanoylglycine. N-Hexanoylglycine has a mass (m/z value of the [M-H]⁻ ion) according to ion mass spectrometry of about 267.0774.

In other embodiments, the biomarkers of the present invention for determining gamma radiation exposure by an animal or human, may also include xanthine base, ion, salt, or any combination thereof. Specific examples of suitable xanthine base biomarkers include xanthosine, 2′-deoxyxanthosine, or any ion or salt, or any combination thereof.

Another biomarker is the compound 2′-deoxyxanthosine, which has a mass (m/z value of the [M-H]⁻ ion) of about 267.0774. This biomarker is in the class of 2′-deoxynucleosides, more specifically a purine 2′-deoxyriboside. The purine riboside equivalent of 2′-deoxyxanthosine, xanthosine, is another biomarker according to the present invention, as well as xanthine, the base from which both xanthosine and 2′-deoxyxanthosine are derived.

In sum, β-thymidine, 2′-deoxyuridine, and 2′deoxycytidine are all pyrimidine nucleosides, specifically, pyrimidine 2′-deoxyribosides. Xanthine is a purine base. Xanthosine and 2′-deoxyxanthosine are both purine nucleosides, specifically, purine ribosides. All six compounds are metabolites of the nucleic acids DNA and RNA. 3-Hydroxy-2-methylbenzoic acid 3-O-sulfate can originate as metabolite of the gut flora, indicative of this as a target of ionizing radiation. Taurine is a marker of liver damage, indicative that the liver may be a target for ionizing radiation damage. N-Hexanoylglycine is a metabolite of fatty acid metabolism, probably in the liver and therefore also indicating that the liver may be a target of ionizing radiation at the doses employed.

In other embodiments, the biomarkers of the present invention may also be characterized as being a specific metabolite in an animal or human when exposed to a particular dose of gamma radiation. Analysis of the biomarkers can determine the gamma radiation dose received by an animal or human. The gamma radiation dose can be in the range of from non-lethal levels, generally about 3 Gy and below for mice, up to lethal levels, generally about 8 Gy and above for mice. The biomarkers of the present invention arise within an animal or human when dosed with gamma radiation generally in the range of from about 1 Gy to about 10 Gy. Typically, the biomarkers arise when the animal or human receives a dose in excess of about 1 Gy, about 2 Gy, about 3 Gy, about 4 Gy, about 5 Gy, about 6 Gy, about 7 Gy, or even about 8 Gy.

The present invention also provides methods for determining gamma radiation exposure by an animal or human. These methods can be carried out by first collecting a biological fluid from the animal or human and measured (i.e., determined) the amount of one or more biomarkers specific to gamma radiation in the biological fluid. Suitable biomarkers may include a pyrimidine base, a purine base, a xanthine base, a pyrimidine nucleoside, a purine nucleoside, a xanthine nucleoside, a metabolite of a nucleic acid, a metabolite of fatty acid metabolism, or any salt, ion, or combination thereof. Suitable specific biomarker examples include 3-hydroxy-2-methylbenzoic acid 3-O-sulfate, N-hexanoylglycine, β-thymidine, taurine, xanthine, xanthosine, 2′-deoxyuridine, 2′-deoxycytidine, or any salt, ion or combination thereof.

The amount of biomarkers can be measured using any of a variety of methods known in the art of analytical chemistry. Suitable measurement methods include chromatography, mass spectrometry, differential ion mobility spectroscopy, radioimmunoassay, nuclear magnetic resonance, infrared spectroscopy, visible spectroscopy, ultraviolet spectroscopy, immunological assay, colorimetric assay, Raman spectroscopy, capillary electrophoresis, or any combination thereof. Preferably, ultra-performance liquid chromatography—time-of-flight mass spectrometry is used to determine the amount of the biomarkers.

The methods can be applied to any of a variety of biological fluids. Suitable biological fluids include whole blood, blood plasma, blood serum, urine, breast milk, mucus, saliva, interstitial fluid, lymph, tears, sweat, sebum, semen, prostatic fluid, vaginal secretion, ear wax, or any combination thereof. Preferably, the biological fluid is obtained non-invasively, such as urine, saliva, tears, sweat, sebum or ear wax. Most preferably the biological fluid that is tested is urine.

A wide range of biomarkers can be tested using any of these analytical chemistry methods. Preferred analytical chemistry methods are selected to be able to measure the amount of the biological biomarker at a concentration in the range of from 1 pg/μl to 5000 pg/μl, or even from 2 pg/μl to 2500 pg/μl, or even from 5 pg/μl to 1000 pg/μl, based on volume of the biological fluid.

After the amount of the one or more biomarkers is measured, the method for determining the gamma radiation exposure by an animal or human includes correlating the amount of the measured one or more biomarkers to the amount of gamma radiation exposure by the animal or human. The method steps of first collecting a biological fluid from the animal or human, then measuring the amount of one or more biomarkers specific to gamma radiation in the biological fluid, and correlating the amount of said biomarkers to the amount of gamma radiation exposure by the animal or human, can be carried out on a cancer patient who is undergoing, or has received, radiation treatment. These steps can be carried out iteratively for treating a cancer patient in order to maximize the likelihood of achieving a successful outcome. For example, the dose of radiation used to treat the cancer patient can be controlled by the amount of one or more biomarkers specific to gamma radiation measured in the biological fluid. If a threshold limit for any one or more of the biomarkers described herein is measured with the patients biological fluid, then it may indicate a need to reduce the level of radiation received by the cancer patient. In contrast, measuring too low a level of a biomarker in a cancer patient may indicate that the patient may not be receiving a high-enough gamma radiation dose. Concurrent treatment and biological marker testing protocols for gamma radiation will improve the ability of medical professionals to treat cancer patients with gamma radiation.

In these methods, the amount of said biomarkers can be correlated to the amount of gamma radiation exposure by the animal or human in any of a variety of ways, for example, by the mathematical manipulation of the concentration of the one or more biomarkers. Concentrations of non-biomarker concentrations, as well as concentrations of tracer or control markers can also be mathematically manipulated. Any type of mathematical manipulation can be used, for example by applying the functions of addition, subtraction, multiplication, and division to the biomarker concentrations, non-biomarker concentrations, tracer concentrations, and control concentrations. For example, the mathematical manipulation of division is used to determine the ratio of the concentration of two or more of the biomarkers. In this embodiment, the ratio of the concentration of the two biomarkers can be in the range of from 1:10,000 to 10,000:1, or even in the range of from 1:1,000 to 1,000:1, or even in the range of from 1:100 to 100:1, or even in the range of from 1:10 to 10:1, or even in the range of from 1:2 to 2:1, or any combination thereof. These mathematical manipulations may further comprise the manipulation of one or more constants, such as in a polynomial expansion:

S=A₀+A₁B₁+A₂B₂²+A₃B₃³+A₄B₄⁴+ . . . AₙBₙⁿ

wherein,

S is correlated to the amount of gamma radiation exposure by the animal or human;

Ai, wherein i=0 to n, represents a constant;

Bi, wherein i=0 to n, represents the concentration of one or more biomarkers;

n is an index number, or exponent.

Systems for determining gamma radiation exposure of an animal or human includes a sample introduction section for collecting a fluid sample comprising one or more biomarkers for gamma radiation. The collected fluid sample is then volatilized in a volatilization section. There is also provided an ion source for ionizing a portion of the volatilized sample, and an ion mobility based filter for focusing at least one biomarker. Suitable ion mobility filters are capable of isolating an ion of a pyrimidine base, a purine base, a xanthine base, a pyrimidine nucleoside, a purine nucleoside, a xanthine nucleoside, a metabolite of a nucleic acid, a metabolite of fatty acid metabolism, or any salt, ion, or combination thereof. Preferably, the ion mobility based filter is capable of isolating an ion of 3-hydroxy-2-methylbenzoic acid 3-O-sulfate, N-hexanoylglycine, β-thymidine, taurine, xanthine, xanthosine, 2′-deoxyuridine, 2′-deoxycytidine, or any combination thereof. Finally, the system includes a detector for detecting the at least one biomarker.

In the systems of the present invention for determining gamma radiation exposure of an animal or human, the biomarker ions are capable of passing through the ion mobility filter in such a fashion that biomarker ions in the sample flows through an asymmetric field. For example, the ion mobility filter can be configured to apply a compensation field to the asymmetric field to selectively pass ions through the filter. To selectively pass ions through the filter, system may incorporate an electronic controller for controlling at least one condition of the filter. For example, the controller can be configured for storing information about filter conditions associated with filtering at least one known biomarker and adjusting the filter conditions to enable the at least one known biomarker to pass through the asymmetric field. In alternative embodiments, the controller can be configured for storing information about filter conditions associated with filtering a plurality of known markers and scanning the filter conditions to enable the plurality of known markers to pass through the asymmetric field.

The systems for determining gamma radiation exposure of an animal or human may incorporate a gas chromatograph from which a portion of the sample is eluted before one of ionizing and pass through the ions. In other embodiments, the systems may further include a pre-filter for filtering the sample using a membrane, such as a polymer membrane. Suitable polymer membranes include a perfluorinated polymer, a silicone, a polyolefin, or any combination thereof. Likewise, addition embodiments of the systems of the present invention may further include one or more pro-filters for removing unwanted components before, during or after sample volatilization.

Further details concerning the fabrication of systems for determining gamma radiation exposure in biological fluids can be found in U.S. Pat. No. 7,355,170, “Systems for differential ion mobility analysis”, to Miller et al., issued Apr. 8, 2008, the entirety of which is incorporated by reference herein.

Animals or humans exposed to high amounts of gamma radiation can also be treated according to the methodologies described herein. These methods include first collecting a biological fluid from the animal or human and measuring the amount of one or more biomarkers specific to gamma radiation in the biological fluid to develop a radiation exposure profile of the animal or human. Any type of biological is envisioned as being collected and measured, with the preferred biological fluids capable of being collected non-invasively, such as urine, and others described herein above. After the radiation exposure profile is generated, the radiation exposure profile is correlated to one or more compounds capable of counteracting the metabolic effects of the gamma radiation on the animal or human. The radiation exposure profile suitably correlates the one or more biomarkers to a change in the redox state of the cell. For example, the change in the redox state of the cell can arise from a generation of reactive oxygen species arising from the radiolysis of water molecules. Alternatively, the change in the redox state of the cell may reduce enzymic reactions that are dependent upon one or more reduced nucleotide cofactors, flavin cofactors, or any combination thereof. Suitable reduced nucleotide cofactors or flavin cofactors may include NADH, NADPH, FADH2, or any combination thereof. In another embodiment, the change in the redox state of the cell can increase enzymic reactions that are dependent upon one or more oxidized nucleotide, flavin cofactors, or any combination thereof. In this embodiment, the one or more oxidized nucleotide cofactors or flavin cofactors can include NAD, NADP, FAD, or any combination thereof.

Ionizing radiation cellular toxicity most likely involves the generation of reactive oxygen species (ROS) through the radiolysis of water molecules. ROS then can deplete antioxidant defenses, such as glutathione, etc. and push the cell to a “pro-oxidant state”. By this alteration of the redox state of the cell, enzymic reactions that are dependent upon reduced nucleotide and/or flavin cofactors (such as NADH, NADPH, FADH2) will be compromised, while reactions that depend on oxidized nucleotide and/or flavin cofactors (NAD, NADP, FAD) can be hastened. Hundreds of such biochemical pathways are likely to be so affected by the presence of ROS.

Finally, in response to generation of the radiation exposure profile, the method includes administering one or more compounds to the animal or human. In this regard, some symptoms of high gamma radiation exposure (i.e. radiation sickness) can be related to the biomarkers according to the present invention, and thus knowledge of these biomarkers can guide therapy. Accordingly, the bolstering of anti-oxidant defenses through the administration of N-acetylcysteine, can be suitably used.

### Examples and Additional Illustrative Embodiments

The search for biomarkers of effective dose and early effect of ionizing radiation exposure in both humans and experimental animals has a history spanning several decades. Blood cells and serum have proven to be abundant sources of human radiation biomarkers, including those of DNA damage and repair, chromosomal aberrations, DNA-protein crosslinks, red blood cell polyamine levels, serum proteomic profiles, and gene expression profiles determined by both microarrays and RT-PCR.

Urine has also furnished insights into metabolic perturbations associated with radiation exposure. Urine analysis has the added advantage of giving a metabolic picture over time because it accumulates in the bladder and can be collected and pooled over set periods, as opposed to the snapshot obtained from a single blood sample. Historically, efforts have concentrated largely on neurotransmitters and their metabolites, on the premise that stress, including radiation stress, should trigger the release of neurotransmitter molecules. Examples include 5-hydroxyindoleacetic acid, indoxyl sulfate, 3-methoxy-4-hydroxymandelic acid, 3-methoxy-4-hydroxyphenylglycol, metanephrine, normetanephrine, and homovanillic acid. More recently, urinary markers of DNA damage and repair have been determined, including thymine glycol, thymidine glycol, and 8-hydroxyguanine. Other potential urinary biomarkers of ionizing radiation that have been reported include thromboxane B₂ and 8-iso-prostaglandin F_{2α}, although this latter example is controversial.

Many of these aforementioned studies were carried out in laboratory rodents. The approaches to uncovering biomarkers for ionizing radiation damage have all been predicated on known or suspected biological effects of radiation, such as neurotransmitter release, DNA damage or inflammation. Given the considerable number of biological molecules involved in just these processes, the number of reports of different biomarkers of ionizing radiation has been modest. One report, however, investigated possible radiation injury with no prior hypothesis except that there should be detectable in urine signs of disturbances in cellular metabolism caused by radiation exposure following the Chemobyl reactor incident. ¹H and ³¹P NMR analysis of human urines revealed some poorly defined changes in “N-trimethyl groups” and in creatinine, citric acid, glycine and hippuric acid.

Perusal of the pathways of cellular metabolism reveals that the small molecular weight (<600 Da) intermediates and end-products of metabolism that are excreted in the urine are mainly acids, phenols, phenolic acids, and amino acids. Purely basic compounds are not in general excreted in urine without metabolism to acidic or Zwitterionic metabolites. Therefore, urine contains a host of anionic substances, with humans excreting about 60 mmol organic acids per day with a mean urinary pH of approx. 6.0. Metabolomics is a means of measuring small-molecule metabolite profiles and fluxes in biological matrices, following genetic modification or exogenous challenges, and is an important component of systems biology, complementing genomics, transcriptomics and proteomics. The ability to register the increases and decreases in intermediary metabolites has progressed considerably due to advances in analytical chemical platforms for metabolite detection and quantitation, and in chemometric software for performing multivariate data analysis on very large data sets. As such, metabolomics is able to provide an unbiased evaluation of upward and downward metabolite fluxes.

In the examples and further embodiments described herein, the high resolution capability of ultra-performance liquid chromatography (UPLC), coupled with the accurate mass determination of time-of-flight mass spectrometry (TOFMS) and various multivariate data analyses (MDA) was used to uncover radiation metabolomic responses in mice γ irradiated with either 3 or 8 Gy. Such radiation metabolomic signatures are useful in designing protocols and novel methodologies for screening at-risk human populations for measures of radiation dose exposure.

Materials and Methods

Compounds. The following compounds were obtained from Sigma-Aldrich Co., St. Louis, Mo., xanthine, xanthosine, 2′-deoxyuridine, 2′-deoxycytidine, taurine, theophylline, 4-nitrobenzoic acid, creatinine, α-thymidine, β-thymidine, 2-, 3-, and 4-hydroxyphenylacetic acid, mandelic acid (α-hydroxyphenylacetic acid), 3-, 4-, and 5-methylsalicylic acid, 3-hydroxy-4-methylbenzoic acid, 4-hydroxy-2-methylbenzoic acid, and 4-hydroxy-3-methylbenzoic acid. 6-Methylsalicylic acid (2,6-cresotic acid), 3-hydroxy-2-methylbenzoic acid (3,2-cresotic acid), 3-hydroxy-5-methylbenzoic acid (3,5-cresotic acid), and 5-hydroxy-2-methylbenzoic acid (5,2-cresotic acid) were obtained from Drug Synthesis and Chemistry Branch, Developmental Therapeutics Program, Division of Cancer Treatment and Diagnosis, National Cancer Institute. Stachadrine hydrochloride (N-methylproline, proline betaine) was obtained from Extrasynthese (Lyon, France) and N-hexanoylglycine was purchased from the Metabolic Laboratory, VU Medical Center (Amsterdam, Netherlands). 2′-Deoxyxanthosine was the kind gift of Dr. Peter Dedon, MIT. All inorganic reagents and solvents were of the highest purity obtainable.

Generation of O-sulfate conjugates in situ. 2-, 3-, and 4-Hydroxyphenylacetic acid, mandelic acid, 3-, 4-, and 5-methylsalicylic acid, 3-hydroxy-4-methylbenzoic acid, 4-hydroxy-2-methylbenzoic acid, 4-hydroxy-3-methylbenzoic acid, 6-methylsalicylic acid (2,6-cresotic acid), 3-hydroxy-2-methylbenzoic acid (3,2-cresotic acid), 3-hydroxy-5-methylbenzoic acid (3,5-cresotic acid), and 5-hydroxy-2-methylbenzoic acid (5,2-cresotic acid) were all treated individually with conc. H₂SO₄ (sp. gravity 1.86) in order to generate in situ their respective O-sulfate conjugates. Experiments were designed in an attempt to minimize the extent of aromatic ring sulfonation by sulfating each acid (2-5 mg) in conc. H₂SO₄ containing 10% water, both on ice and at room temperature. Aliquots were taken at 30, 60, 90, and 120 min and neutralized on ice by the careful addition of ammonium hydroxide solution. Resulting neutral solutions were analyzed by UPLC-TOFMS (see below).

Animals. Male C57BL\6 mice at 11-20 weeks of age, obtained from Charles River Laboratories, Inc. (Wilminton, Mass.) by way of NCI-Frederick (Frederick, Md.), were used for this study. This strain is intermediate in radiation sensitivity, with 3-4 month old male mice having a reported 30 day mean LD50 of 6.5 Gy, which compares to <5.7 and 7.3 Gy in the most radiosensitive and radioresistant strains studied, BALB/cJ and 129/J, respectively. Mice were watered and fed NIH31 chow ad libitum and housed under a standard 12 h light/12 h dark cycle. All animal handling and experimental protocols were designed for maximum possible well-being, conformed to the guidelines stipulated by the National Institutes of Health Office of Animal Care and Use, and were approved prior to the initiation of this study.

Radiation Dosing. Groups of mice were exposed to doses of 3 (n=12), 6 (n=8), 7 (n=10), 8 (n=10 and 12), or 11 Gy (n=10) γ radiation emitted from a ¹³⁷Cs source in a Mark I Model 68 small animal irradiator (JL Shepherd & Associates, San Fernando, Calif.) operating at 2.57 Gy/min. The Mark 1 has a single ¹³⁷Cs source and provides uniform doses to small animals centered on the revolving turntable revolving at a constant rate of 4.75 per minute within the chamber. Mice were irradiated in a pie cage (25.5 cm inner diameter) that separates and evenly distributes them for uniform exposures. The source-to-surface distance in this configuration varies over time at a constant rate, averaging 20 cm with a range of 7.3 to 32.8 cm. The dose for a given mouse is uniform and virtually identical to the dose of each cagemate. Physical doses within the chamber were assessed using models AT-742 (0-2 Gy) and AT-746 (0-6 Gy) direct reading dosimeters (Arrow-Tech, Inc., Rolla, N. Dak.).

A dose of 7 Gy in mice is about equivalent to 4 Gy for humans. Thus, the mouse doses employed were roughly equivalent to doses of 1.7 and 4.6 Gy to humans. That is, the doses were either below or within the range (2.5-5 Gy) considered to be associated with the radiation hematopoietic syndrome. For identification of urine biomarkers, doses of 3 and 8 Gy were chosen. The LD50/30 for C57BL/6 mice in our laboratories is 7 to 8 Gy so 8 Gy was chosen in order to maximize any potential metabolomic response and 3 Gy as a sublethal dose generally associated with some cellular changes but no outward, noticeable symptoms or behavior changes.

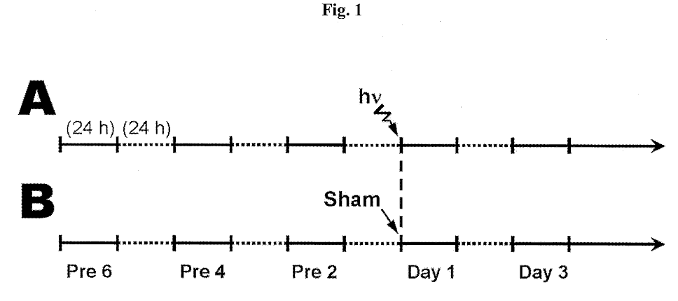

Urine Collection. Urine samples from mice housed individually in Nalgene metabolic cages (Tecniplast USA, Inc., Exton, Pa.) were collected over continuous 24-h periods with alternate 24-h rest intervals. Urine was collected over 24 hours to avoid the effects of diurnal variation on urine metabolite profiles shown by others. Three 24-h urine samples per mouse were obtained at 6, 4 and 2 days pre-exposure. Control mice were handled identically, including a sham irradiation with exposure to 0 Gy in the irradiator, and were used for simultaneous control urine sample collection. All urine samples were stored at −80° C. until analyzed. The sample collection protocol is illustrated in FIG. 1.

Ultra-Performance Liquid Chromatography—Time-of-Flight Mass Spectrometry (UPLC-TOFMS) Analyses. The urine samples were analyzed by UPLC-TOFMS in order by timepoint and by mouse number in the same, continuous session to eliminate instrument bias as a potential confounder. The operator was blinded to the exposure status of the samples at the time of the UPLC-TOFMS analysis so that confounding by operator bias was minimized. Urine aliquots (50 μl) were diluted 1:5 with 50% aqueous acetonitrile (200 μl) and centrifuged at 13,000×g for 20 min at 4° C. to remove particulates and precipitated proteins. Aliquots (100 μl) of supernatant were transferred to auto-sampler vials for UPLC-TOFMS analysis and injected (5 μl) onto a reversed-phase 50×2.1 mm ACQUITY® 1.7 μm C18 column (Waters Corp, Milford, Mass.) using an ACQUITY® UPLC system (Waters) with a gradient mobile phase comprising 0.1% formic acid solution (A) and acetonitrile containing 0.1% formic acid solution (B). Each sample was resolved for 10 min at a flow rate of 0.5 ml/min. The gradient consisted of 100% A for 0.5 min, 20% B for 3.5 min, 95% B for 4 min, 100% B for 1 min, and finally 100% A for 1 min. The column eluent was directly introduced into the mass spectrometer by electrospray. Mass spectrometry was performed on a Q-TOF Premier® (Waters) operating in either negative-ion (ESI−) or positive-ion (ESI+) electrospray ionization mode with a capillary voltage of 3000 V and a sampling cone voltage of 30 V. The desolvation gas flow was set to 650 L/h and the temperature set to 350° C. The cone gas flow was 50 L/h, and the source temperature was 120° C. Accurate mass was maintained by introduction of LockSpray® interface of sulfadimethoxine (309.0658 [M-H]⁻) at a concentration of 250 pg/μl in 50% aqueous acetonitrile and a rate of 30 μl/min. Data were acquired in centroid mode from 50 to 800 m/z in MS scanning. Tandem MS collision energy was scanned from 5 to 35 V.

Data Processing and Multivariate Data Analysis. Centroided and integrated mass spectrometric data from the UPLC-TOFMS were processed to generate a multivariate data matrix using MarkerLynx® (Waters). All data for each urine ion were normalized by relative creatinine concentrations on a per sample basis. Centroided data were Pareto-scaled and further analyzed by principal components analysis (PCA) and orthogonal partial least squares (OPLS) using SIMCA-P+ software (Umetrics, Kinnelon, N.J.). Samples were classified as either from control (y=0) or irradiated (y=1) mice for OPLS used to determine which metabolites contribute most to the separation in the scores space and are thus elevated in urine samples from irradiated mice compared with samples from control mice. Selection of candidate markers was accomplished by examining the scatter S-plots of significance (P) vs. weight, i.e. how much a particular ion correlated to the model and a measure of its relative abundance. In each loadings S-plot the ions positioned most distant from the origin in the upper right quadrant were interrogated for consideration as a candidate marker. Twenty-two ions were then chosen based on S-plot coordinates and lowest P values derived from both two-tail t tests (parametric) of the normalized means and Wilcoxon-Mann-Whitney tests (nonparametric) of the normalized data. Data were also analyzed using the random forests machine learning algorithm to classify urines samples as irradiated or non-irradiated. Random forest variable importance scores were used to estimate the usefulness of each model variable. In order to derive reproducible variable importance scores, a panel of twenty five independent random forest models, each with 10,000 trees, was trained on the set of all ions (ESI⁺ and ESI⁻). Next, the importance score rank for each variable in each model was computed. The ranks were then averaged over the set of models. Bootstrapping the results from the 25 independent random forests was used to determine the 95% confidence intervals of the variable importance ranks. The minimal number of variables necessary to build an optimal random forest model for classifying each group was determined with a “greedy” (i.e., locally optimal but potentially globally suboptimal) approach. The average performance of sets of 25 random forest models each trained using a subset of the top ranked variables were compared. The set of 25 random forest models were trained using the previously determined top 10, 20, 50, 100, 150, 250, 350, 500, 750, or 1000 most important model variables. The set of models that achieved the best average classification performance was concluded to contain the most meaningful set of variables. Bootstrapping of the 25 forests was used to determine the variation in model performance within each of the sets of 25 models. In the case that more than one set of top performing variables was non-statistically significantly better than other models, the best model was the one that used the most variables. A cohort of ions (20-30) was chosen in each experiment to characterize the similarities and differences in the responses to these two doses. Elemental compositions were generated with MarkerLynx based on the exact masses of the high-contribution score metabolites. Identification of the top metabolites was informed by biological relevance and likelihood of presence in the urine. Authentic standards at 20-60 μM in 50% acetonitrile and 0.1% formic acid were then used to confirm the identities of the markers with UPLC-MS/MS. Tandem MS (MS/MS) fragments the molecules in a consistent manner. Therefore, putative urine metabolites provide a MS/MS fragmentation spectrum identical to the fragmentation spectrum of the known standards.

Quantification and statistical analysis of urine biomarkers. QuanLynx software (Waters) was used to quantify urine metabolites based on their peak areas. Calibration curves were constructed for authentic creatinine (MH⁺ 114.0667 m/z), thymidine ([M-H]⁻241.0824 m/z), N-hexanoylglycine ([M-H]⁻172.0974), and taurine ([M-H]⁻124.0068) duplicate standards in 50% aqueous acetonitrile at concentrations ranging from 0.19 to 100 μM. Theophylline (0.5 μM; MH⁺ 181.0726 m/z) and 4-nitrobenzoic acid (3 μM; [M-H]⁻166.0141) were included as internal standards. Quantitation was accomplished using absolute peak area ratios (MH⁺, analyte/theophylline; [M-H]⁻, analyte/4-nitrobenzoic acid) over standard concentrations for linear regression analysis. Calibration curves were linear for each analyte (r², P) as follows: creatinine (0.96, <0.0001), β-thymidine (0.95, <0.001), N-hexanoylglycine (0.98, <0.0001), taurine (0.98, <0.0001). Analyte concentrations in mouse urine (diluted 5- to 400-fold in duplicate) were determined from the respective calibration curves. β-Thymidine, N-hexanoylglycine, and taurine are expressed as μmol/mmol creatinine (normalized). All samples and standards were run in duplicate, and the resultant concentrations were averaged. Analyte concentrations were tested for normal distribution by the skewness and kurtosis test. Mean concentrations of β-thymidine were tested for difference according to exposure status by a two-tailed t test assuming unequal variances (α=0.05; variance ratio test P<0.05). Mean concentrations of N-hexanoylglycine in the 3 Gy experiment were tested for difference by the Mann-Whitney U test because at least one group in the comparison was not normally distributed. For the 8 Gy experiment, at test assuming equal variances was used under the same parameters already mentioned. Mean taurine concentrations were tested using at test assuming equal variances with the same parameters mentioned otherwise for the 3 Gy experiment and the Mann-Whitney U test for the 8 Gy experiment. Fold-change in analyte concentrations were found by transforming the data to log base 2 followed by a two-tail t test. The mean differences and corresponding 95% confidence intervals were then used to calculate fold change using 2^{x} where x=mean difference, lower value of the confidence interval, or upper value of the confidence interval. Additionally, potential confounding variables, namely body weight and urine sample volume were also examined for exposure-specific differences. Mean sample volumes were compared by two-tailed t tests assuming equal variances as described above. For 3 Gy, mean body weights were compared by Mann-Whitney U test, whereas for 8 Gy the means were compared by t test assuming unequal variances. All statistical analyses were performed using STATA (Stata Corp LP, College Station, Tex.). Graphical presentations of data were prepared using Prism (GraphPad Software, Inc., San Diego, Calif.).

Bioinformatics. Gene Expression Dynamics Inspector (GEDI) (G. S. Eichler, S. Huang and D. E. Ingber, Gene Expression Dynamics Inspector (GEDI): for integrative analysis of expression profiles. Bioinformatics 19, 2321-2322 (2003); Y. Guo, G. S. Eichler, Y. Feng, D. E. Ingber ad S. Huang, Towards a holistic, yet gene-centered analysis of gene expression profiles: a case study of human lung cancers. J Biomed Biotechnol 2006, 69141 (2006) was used for analysis and visualization of patterns in the MarkerLynx data matrices. The software package was developed for, and applied in the past to, the interpretation of gene expression data. GEDI creates intuitive visualizations of each sample based on the Self-Organizing Map (SOM) algorithm. However, it improves the interpretability of typical SOMs by rendering the output for each experimental sample as a two-dimensional heatmap-like mosaic of colored tiles. GEDI starts by training a conventional SOM to assign each ion to a mosaic tile in such a way that ions with similar patterns across the samples are placed in the same or nearby tiles. After that training, GEDI, unlike the conventional SOM algorithm, creates a series of coherent mosaic heatmaps representing each sample's overall ion profile. The GEDI analysis here used Pearson's correlation as the similarity metric in training of the SOM. In addition, to identify the common expression patterns within each dose group, GEDI was used to compute average mosaics.

### Results

Influence of Cage Stress of the Mouse Urinary Metabolome. Preliminary investigations determined that the handling and caging of all mice influenced their urinary metabolome (data not shown). In particular, PCA revealed the elevation of a particular urinary constituent that at first was believed to be due to the stress of housing of individual mice in metabolic cages with metallic mesh floors. This urinary constituent was elevated up to two-fold during the second day in the metabolic cage environment, but dropped back to baseline levels during a fourth day in the metabolic cage. The ion in question had a retention time of 0.40 min and m/z=144.1020 in positive ion mode, corresponding to an empirical formula for the protonated molecular ion of C7H14NO2 with 3.5 ppm error. Both co-chromatography with an authentic standard and tandem MS fragmentation spectra established that this ion is derived from stachydrine (proline betaine, N-methylproline) in urine. Stachydrine is found in alfalfa where it is synthesized from ornithine and therefore is likely a constituent of the laboratory animal chow. Extraction of the NIH31 diet with water/acetonitrile mixtures confirmed the presence of stachydrine in the diet. Therefore, without being bound by any limitation or theory of operation, it is proposed that the increased urinary excretion of stachydrine during the first three stints in metabolic cages is a simple reflection of increased feeding and not attributable to psychological stress response per se. Stachydrine is biotransformed in rats to various oxidized and conjugated urinary metabolites and presumably the same is true in mice. Accordingly, in all experiments, mice were first acclimated to the metabolic cages on three prior occasions as shown in FIG. 1, to minimize this dietary/stress effect on the urinary metabolome.

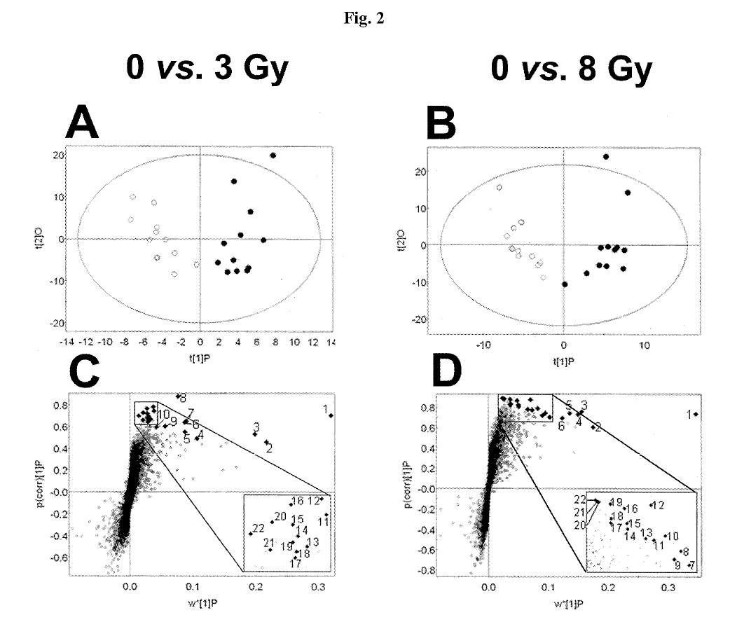

Metabolomic Analysis of Urine after 3 Gy γ Irradiation. Mice were irradiated with 3 Gy (n=12) and sham irradiated (n=12) and urines collected in metabolic cages for 0-24 h. Irradiated mice appeared to have smaller urine volumes (0.84±0.62 ml, mean±s.d.) than sham irradiated mice (1.20±0.79 ml) but this difference was not statistically significant. UPLC-TOFMS analysis of urines revealed a large data matrix containing approximately 6,000 negative ions per mouse urine, which was subjected to both PCA and OPLS multivariate data analyses. Unsupervised PCA did not give a good clustering of the sham and irradiated data sets (data not shown). Therefore, supervised OPLS analysis was performed, whereby the data were classified as either irradiated or sham. FIG. 2A shows an OPLS scores plot for the 3 Gy experiment, depicting a clear separation between mice that were irradiated and sham irradiated. FIG. 2C shows a scatter S-plot from the OPLS analysis of the 3 Gy data. The 22 most outlying ions have been annotated 1 to 22.

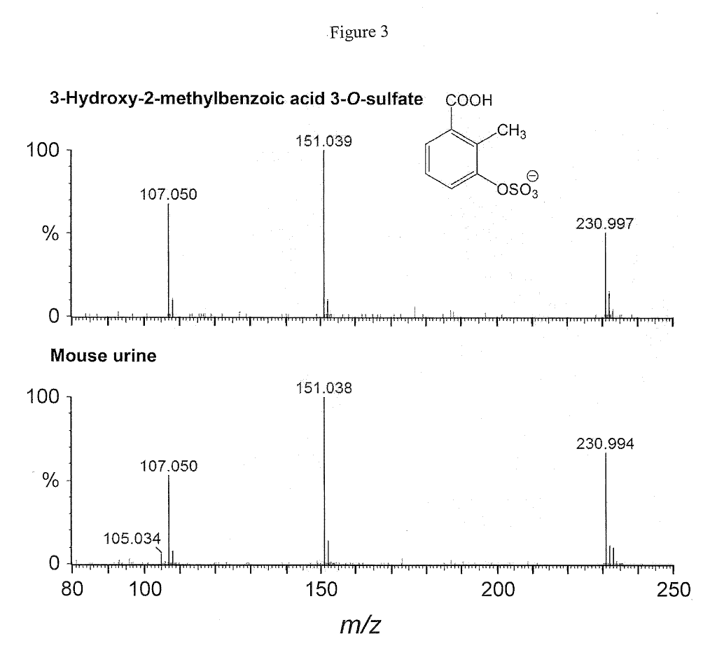

Identification of Urinary Biomarkers after 3 Gy γ Irradiation. Biomarker #1, from the OPLS scatter S-plot in FIG. 2C, with a [M-H]⁻=230.996, gave an empirical formula of C₈H₇O₆S⁻ with a mass error of 1.3 ppm. Treatment of urine with arylsulfatase (Type H-1 from Helix pomatia) caused this ion to completely disappear, confirming that it derived from a sulfate conjugate. The metabolite that is conjugated with sulfate would therefore have an empirical formula of C₈H₈O₃, for which exists 14 possible carboxylic acid candidates, namely, 2-, 3-, and 4-hydroxyphenylacetic acid, mandelic acid, 3-, 4-, 5-, and 6-methylsalicylic acid, 3-hydroxy-2-methyl-, 3-hydroxy-4-methyl-, 3-hydroxy-5-methyl-, 5-hydroxy-2-methyl-, 4-hydroxy-2-methyl-, and 4-hydroxy-3-methyl-benzoic acid. In addition, two aldehydes with the same empirical formula might also be conjugated with sulfate and found in urine, 3-hydroxy-4-methoxybenzaldehyde (isovanillin) and 4-hydroxy-3-methoxybenzaldehyde (vanillin). However, it has long been established that these aldehydes are largely oxidized to their respective benzoic acids prior to urinary excretion by laboratory animals (38, 39). Thus, the investigation of the sulfate metabolite was restricted to the 14 aforementioned organic acids. Details of the mass fragmentation of deprotonated molecular ions ([M-H]⁻) of each of the sulfated aromatic acids, are shown in Table 1. Inspection of the UPLC retention times and MS/MS fragmentation patterns readily revealed that the urinary sulfate with [M-H]⁻=230.9962 could not be a phenylacetic acid or salicylic acid derivative. However, the sulfate of 3-hydroxy-2-methylbenzoic acid co-chromatographed with the urinary peak and had an identical fragmentation pattern (FIG. 3). The metabolic origin of 3-hydroxy-2-methylbenzoic acid 3-O-sulfate is not known.

Co-chromatography and tandem MS with authentic standards demonstrated unequivocally that the identity of the biomarker (Table 2), with a [M-H]⁻=172.0985 m/z (C8H14NO3⁻, mass error=6.4 ppm) and retention time of 3.66 min, was N-hexanoylglycine. There were no other high-ranking ions derived from this ion (isotopes, in-source fragments, adducts, dimers).

The identity of the biomarker, with a [M-H]⁻=241.0820 m/z (C10H13N2O5⁻, mass error=1.7 ppm) and retention time of 1.90 min, was thymidine. This was confirmed by tandem MS experiments. However, there are two epimers of thymidine, β-thymidine, the nucleoside present in DNA, and α-thymidine which can be formed in DNA in situ by oxidative stress in the nucleus and removed by nucleotide excision repair. These did not resolve by UPLC, with α- and β-thymidine having retention times of 1.94 and 1.92 min, respectively. Moreover, both epimers had identical tandem MS spectra, with ions of nominal m/z (% abundance) of 241 (100) and 151 (10). Therefore, a longer (150 mm) UPLC column was employed for their analysis, which resolved α-thymidine (3.63 min) from β-thymidine (3.56 min) with a 90% peak-to-peak valley. Additionally, when α-thymidine was added to urine, the extracted ion chromatogram (m/z 241.0824) showed two peaks. When urine was spiked with β-thymidine, only one peak was observed. It was concluded that this biomarker was β-thymidine. There were no other high-ranking ions (isotopes, in-source fragments, adducts, dimers) derived from this ion.

The biomarker with a [M-H]⁻=417.1143 m/z (C10H13N2O5⁻, mass error=1.7 ppm), which would match to a glucuronide of thymidine. However, this biomarker was of low abundance and experiments with β-glucuronidase hydrolysis were inconclusive. Moreover, a glucuronide of thymidine has never been reported. The identity of this biomarker was not further pursued.

The biomarker with a [M-H]⁻=267.0741 m/z (C10H11N4O5⁻, mass error=4.1 ppm) and a retention time of 1.74 min was 2′-deoxyxanthosine as demonstrated by co-chromatography with authentic standard and identical tandem MS fragmentation.

The biomarker with a [M-H]⁻=283.0700 m/z (C10H11N4O6⁻, mass error=7.4 ppm) and a retention time of 1.86 min was xanthosine as demonstrated by co-chromatography with authentic standard and identical tandem MS fragmentation.

The biomarker with a [M-H]⁻=151.0270 m/z (C5H3N4O2⁻, mass error=9.3 ppm) and a retention time of 0.65 min was xanthine as demonstrated by co-chromatography with authentic standard and identical tandem MS fragmentation.

The biomarker with a [M-H]⁻=227.0660 m/z (C9H11N2O5⁻, mass error=3.5 ppm) and a retention time of 1.15 min was 2′-deoxyuridine as demonstrated by co-chromatography with authentic standard and identical tandem MS fragmentation.

The biomarker with a [MH]⁺=228.1000 m/z (C9H12N3O4⁺, mass error=7.0 ppm) and a retention time of 0.54 min was 2′-deoxycytidine as demonstrated by co-chromatography with authentic standard and identical tandem MS fragmentation.

Thus, eight biomarkers for mouse γ irradiation with 3 Gy were unequivocally identified as and 3-hydroxy-2-methylbenzoic acid 3-O-sulfate, N-hexanoylglycine, and β-thymidine, xanthine, xanthosine, 2′-deoxyxanthosine, 2′-deoxyuridine and 2′-deoxycytidine.

Metabolomic Analysis of Urine after 8 Gy γ Irradiation

When mice were irradiated with 8 Gy (n=12) and sham irradiated (n=12), urines volumes also appeared to be smaller in the irradiated animals (0.77±0.58) than the sham irradiated mice (1.03±0.46). As with 3 Gy, this difference did not reach statistical significance. UPLC-TOFMS analysis produced a data matrix of approximately 6,000 ions that was analyzed by PCA and this also did not give a good clustering of irradiated and sham-irradiated mice (data not shown). However, OPLS analysis showed a clear separation and clustering of the groups in the scores plot (FIG. 2B). FIG. 2D displays the scatter S-plot from the OPLS analysis of the 8 Gy data, with the top 22 outlying ions annotated 1 to 22.

Identification of Urinary Biomarkers after 8 Gy γ Irradiation. Of the cohort of principal ions, the biomarker (Table 3) with a [M-H]⁻=172.0985 m/z (C8H14NO3, mass error=6.4 ppm) and retention time of 3.66 min, was N-hexanoylglycine. The identity of the biomarker with a [M-H]⁻=124.0080 m/z (C2H6NO3, mass error=9.7 ppm) and retention time of 0.29 min, was taurine, as demonstrated by co-chromatography with authentic standard and identical tandem MS fragmentation. The identity of the biomarker with a [M-H]⁻=241.0820 m/z (C10H13N2O5, mass error=1.7 ppm) and retention time of 1.90 min, was β-thymidine. The identity of the biomarker with a [M-H]⁻=267.0806 m/z (C10H11N4O5⁻, mass error=28.8 ppm) and a retention time of 1.84 min was 2′-deoxyxanthosine. The identity of the biomarker with a [M-H]⁻=283.0695 m/z (C10H11N4O6⁻, mass error=5.6 ppm) and a retention time of 1.80 min was xanthosine. The identity of the biomarker with a [M-H]⁻=227.0664 m/z (C9H11N2O5⁻, mass error=1.8 ppm) and a retention time of 1.15 min was 2′-deoxyuridine. The identity of the biomarker with a [MH]⁺=228.0980 m/z (C9H12N3O4⁺, mass error=1.8 ppm) and a retention time of 0.60 min was 2′-deoxycytidine. Thus, seven biomarkers for mouse γ irradiation with 8 Gy were unequivocally identified as N-hexanoylglycine, taurine, β-thymidine, xanthosine, 2′-deoxyxanthosine, 2′-deoxyuridine and 2′-deoxycytidine.

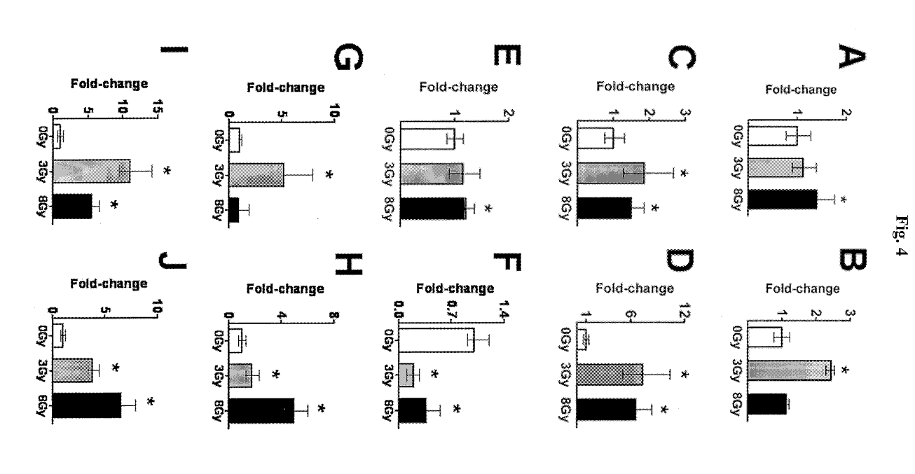

Quantitation of Urinary Biomarkers after 0, 3, and 8 Gy γ Irradiation. The concentration of the discovered biomarkers, β-thymidine, N-hexanoylglycine, taurine, as well as creatinine, were all measured in each urine sample after construction of calibration curves using theophylline (ESI+ mode) and 4-nitrobenzoic acid (ESI− mode) as internal standards. Because no authentic sample of sufficient quantity was available for 3-hydroxy-2-methylbenzoic acid 3-O-sulfate, comparisons of excretion of this biomarker at 0, 3, and 8 Gy doses were made on the basis of relative peak area, normalized to creatinine. Similarly, comparisons of the urinary excretion of xanthine, xanthosine, 2′-deoxyxanthosine, 2′-deoxyuridine and 2′-deoxycytidine were also made on the basis of relative peak area, normalized to creatinine. Urinary creatinine concentration varied widely between groups of mice. Specifically, the 3 Gy irradiated and sham irradiated controls had creatinine concentrations of 3.26±0.91 mM and 2.83±0.56 mM, respectively, which were not statistically significantly different. The 8 Gy irradiated and sham irradiated controls had urinary creatinine concentrations of 1.95±0.51 mM and 1.38±0.33 mM, respectively, which were statistically significantly different (P=0.003). However, when these concentrations were multiplied by urine volumes, the sham irradiated mice excreted a mean of 1.32±0.52 μmol creatinine in 0-24 h, and the 8 Gy irradiated mice a mean of 1.30±0.75 μmol creatinine in 0-24 h, which were not statistically significantly different. Thus, the irradiated mice simply made a small volume of more concentrated urine and 8 Gy irradiation had no apparent effect on the total production and excretion of creatinine. Consequently, biomarkers were then expressed as μmol/mmol creatinine, a variable largely unaffected by urine volume. The urinary creatinine concentrations are displayed in FIG. 4A.

Urinary 3-hydroxy-2-methylbenzoic acid 3-O-sulfate was elevated 2.5-fold after 3 Gy irradiation, but not elevated after 8 Gy radiation (FIG. 4B). N-Hexanoylglycine was elevated 40-80% after γ irradiation (FIG. 4C), from 358±176 to 643±259 μmol/mmol creatinine (P=0.002) for 3 Gy irradiation, and from 556±198 to 802±117 μmol/mmol creatinine for 8 Gy irradiation. β-Thymidine was elevated 6- to 7-fold after γ irradiation (FIG. 4D), from 9.97±4.76 to 67.7±17.4 μmol/mmol creatinine (P<0.001) for 3 Gy irradiation, and from 5.48±1.46 to 35.6±8.84 μmol/mmol creatinine (P<0.001) for 8 Gy irradiation. Urinary taurine was elevated only after the 8 Gy dose (FIG. 4E), sham and 3 Gy values being 7.57±2.36 and 8.52±1.90 mmol/mmol creatinine, respectively. 2′-Deoxycytidine urinary excretion was reduced five-fold after 3 Gy irradiation and three-fold after 8 Gy irradiation (FIG. 4F). Xanthine urinary excretion was elevated five-fold after 3 Gy irradiation, but not after 8 Gy irradiation. Xanthosine urinary excretion was elevated two-fold after 3 Gy irradiation and five-fold after 8 Gy irradiation. 2′-Deoxyuridine urinary excretion was elevated 11-fold after 3 Gy irradiation and five-fold after 8 Gy irradiation. 2′-Deoxyxanthosine was elevated four-fold after 3 Gy irradiation and six-fold after 8 Gy irradiation. Finally, none of the differences in urine volume, creatinine or biomarker excretion could be explained on the basis of different body weights, which were 30.9±1.7, 30.1±2.2, 30.1±1.1, and 31.1±2.3 g for the 3 Gy sham, 3 Gy irradiated, 8 Gy sham, and 8 Gy irradiated groups, respectively. These mean body weights were not statistically significantly different from each other.

Global urinary metabolome changes in response to γ irradiation display a dose-response relationship as viewed by GEDI self-organizing maps

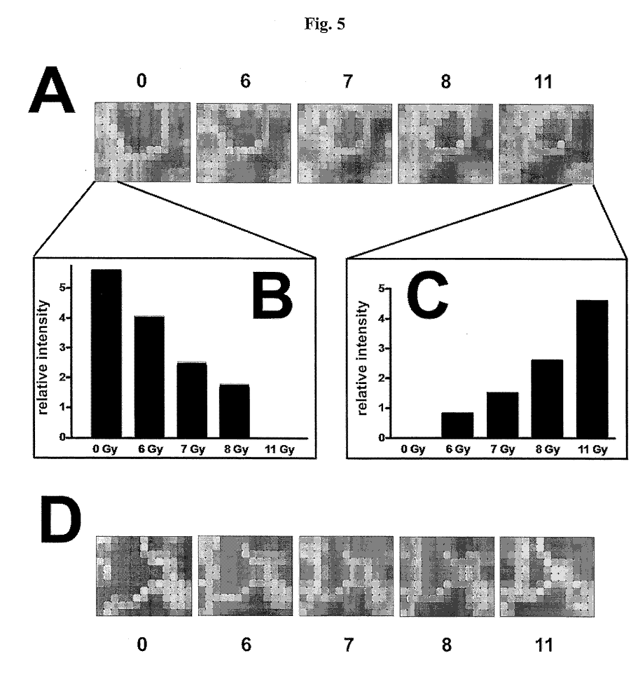

Rather than the analysis of single urinary species, a holistic view of the mouse urinary metabolome was made using GEDI software that was originally designed for analyzing gene expression profiles. This bioinformatics process facilitates visualization of regions of the urinary metabolome that increased and decreased in concentration in response to γ radiation exposure. FIG. 5 shows a series of self-organizing maps for the average urinary metabolomes of groups of mice that had been irradiated with 0, 6, 7, 8, or 11 Gy, first 24 h post-exposure. A clear dose-response relationship exists, both for a group of negative ions in the bottom left-hand corner that decrease in abundance with radiation dose (Panel B) and a group of negative ions that increase in abundance with increasing radiation dose (Panel C). These submatrices of 3×3 cells contain approximately 7.5% of all the ions that comprise the urinary metabolome that is displayed in the 13×11 cell matrix of the self-organizing maps. Moreover, two of the known elevated biomarkers, β-thymidine and N-hexanoylglycine, fall in the bottom right-hand submatrix of 3×3 cells (Panel C). Similar map areas also decreased and increased, respectively, in relative abundance when positive ions were analyzed (Panel D). It is important to note that the positive ions represented in FIG. 5D are distinct urine metabolites from the negative ions shown in FIG. 5A-C, with perhaps only very little overlap with ions that can appear in both negative and positive ionization MS. Together, these results demonstrate, for the first time, a dose-response relationship between γ radiation and biomarkers in the mouse urinary metabolome.

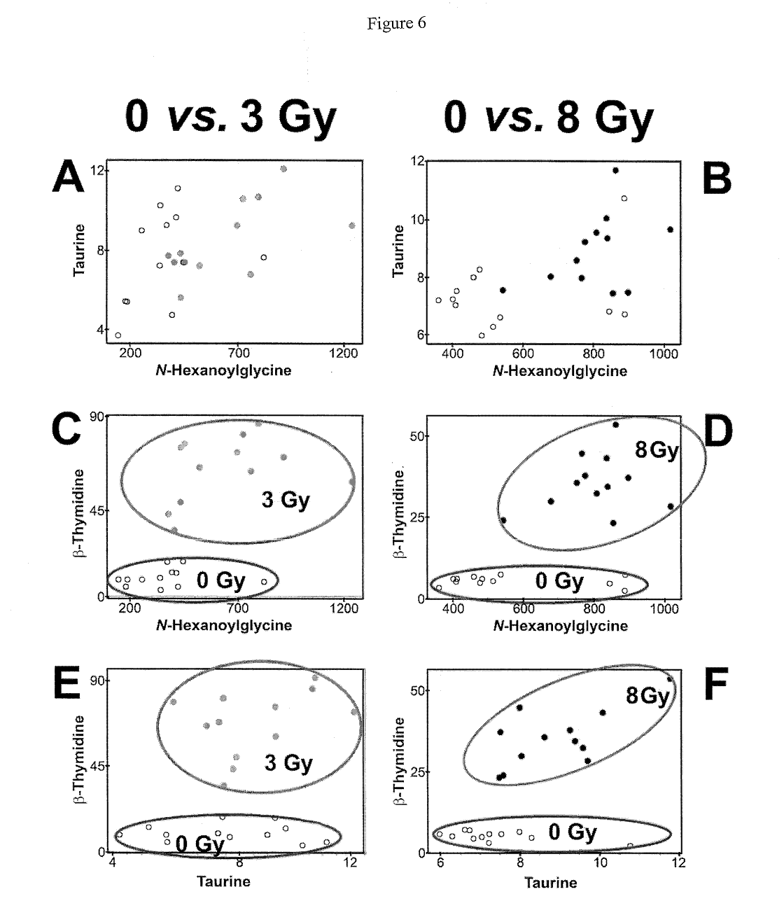

Urinary metabolomic phenotypes for the detection of γ radiation exposure. The combination of pairs of urinary biomarkers, specifically, N-hexanoylglycine and taurine, N-hexanoylglycine and β-thymidine, together with taurine and β-thymidine, were evaluated for their ability to define a urinary metabolomic phenotype that was diagnostic of γ radiation exposure. FIG. 6 displays these phenotypes for both 3 Gy and 8 Gy irradiation versus sham irradiated (control) animals. Plots of N-hexanoylglycine versus taurine were uninformative for both 3 Gy (FIG. 6A) and 8 Gy (FIG. 6B). However, plots of N-hexanoylglycine versus β-thymidine segregated into two phenotypes for 0 versus 3 Gy (FIG. 6C) and 0 versus 8 Gy (FIG. 6D). Additionally, plots of taurine versus β-thymidine segregated into two phenotypes for 0 versus 3 Gy (FIG. 6E) and 0 versus 8 Gy (FIG. 6F). In these models, 3 Gy was not distinguishable from an 8 Gy exposure.

### Discussion

Analysis by UPLC-TOFMS of 24-h urine samples collected immediately following exposure of mice to 3 and 8 Gy γ radiation doses produced data matrices of m/z versus retention time versus normalized ion intensity that, when subjected to multivariate data analysis by OPLS, revealed distinct metabolomic phenotypes for each dose and for sham-irradiated animals (FIG. 2). From the top cohort of ions contributing to this clustering and inter-phenotype separation, a number of urinary biomarkers were unequivocally identified using tandem mass spectrometric comparison with authentic standards. A biological molecule, 3-hydroxy-2-methylbenzoic acid 3-O-sulfate was a biomarker of 3 Gy, together with xanthine, but not 8 Gy exposure. N-Hexanoylglycine, β-thymidine, xanthosine, 2′-deosyxanthosine, 2′-deoxyuridine and 2′deoxycytidine were biomarkers of both 3 Gy and 8 Gy exposure (Tables 2 and 3), all being statistically significantly elevated in urine after irradiation, with the exception of 2′-deoxycytidine which was statistically significantly attenuated after irradiation. Finally, taurine was a biomarker of 8 Gy irradiation only. The change in biomarker urinary concentration in exposed versus sham-irradiated animals was 1.2- to 11-fold. Finally, a clear dose-response relationship in the global view of the urine metabolite profile visualized using GEDI was demonstrated. Of interest is the ratio of, say, 2′-deoxyuridine to 2′deoxycytidine (from which the former may be formed by reactive oxygen or nitrogen species), which was observed to be approximately 55 at 3 Gy and 15 at 8 Gy.

The chemical identities of nine markers were elucidated and confirmed of which six, xanthosine, 2′-desoxyxanthosine, 2′-deoxyuridine, 2′-deoxycytidine, β-thymidine and N-hexanoylglycine, were validated and quantitated across the two experiments. Because these ions were elevated in urine from exposed animals at both doses given independently, these can be concluded to be specific biomarkers of radiation exposure. In addition, 3-hydroxy-2-methylbenzoic acid O-sulfate and xanthine were both found to be statistically significantly elevated in the urine of animals exposed to 3 but not 8 Gy, compared with controls. It was also observed that taurine is statistically significantly elevated in the urine of mice exposed to 8 but not 3 Gy, compared with controls. The point estimate of mean taurine level in the urine from mice exposed to 3 Gy is elevated over that of the controls, albeit not significantly. Without being bound by any particular theory of operation, this is suggestive of a dose-response relationship that will be further explored with doses between 3 and 8 Gy. Accordingly, taurine, xanthine, and 3-hydroxy-2-methylbenzoic acid O-sulfate appear to be validated as biomarkers for gamma radiation. In addition, there are several other ions among the cohort of principal ions highlighted in each experiment that are not common to both experiments. Accordingly, it appears that metabolic profiles are different after lethal versus sublethal γ radiation exposures.

A bioinformatic technique was employed that demonstrated that a large number of urinary constituents co-varied across the sample set with the aforementioned biomarkers. In other words, the 3 Gy and 8 Gy phenotypes that were seen as distinct clusters in the OPLS scores plots (FIGS. 2A and 2B, respectively) arose due to myriad differences in urinary constituents between the irradiated and sham-irradiated animals. This can be seen from the GEDI self organizing maps, where groups of nine interconnected tiles, which represented hundreds of both negative (FIG. 5A) and positive (FIG. 5D) ions, increased (FIG. 5C) in intensity in a dose-dependent manner, while others decreased (FIG. 5B), also in a clear dose-dependent fashion. This is an important proof of principle of radiation metabolomics and also the first time that GEDI self organizing maps have been used to analyze and display global in vivo metabolomic data. These observations indicate the existence of a rich source of additional biomarkers of radiation exposure.

Using pairs of biomarkers (FIG. 6), it may ultimately be possible to predict whether or not a human or animal has been exposed to y radiation and perhaps also the general dose range. It is this approach, refined by the addition of biomarkers, that gives rise to a metabolomics-based protocol for noninvasive radiation biodosimetry in human subjects. Towards this end, Table 4 lists the reported small molecule biomarkers of ionizing radiation exposure for both laboratory animals and humans, together with the calculated m/z values of their protonated and deprotonated molecular ions. It is of note that none of the validated radiation biomarkers of the present invention have been reported previously. The published biomarkers fall into the classes of neurotransmitter metabolites, excised DNA adducts, reactive oxygen products, and general metabolic intermediates. An additional area of future research lies in whether any of the ions listed in Table 4 appear elevated at later timepoints.

The question arises as to the metabolic origins of the novel radiation biomarkers reported here. One of the most dramatic post-irradiation change was in β-thymidine (FIG. 4D), which may reflect increased synthesis, decreased utilization, or elevated renal tubular outward transport. Without being bound by any theory of operation it is also possible that the products of oxidative DNA damage, thymine glycol and thymidine glycol, might be metabolically reconverted to thymidine, although this is known not to occur in E. coli and is therefore unlikely. Interestingly, when [³H]thymidine was administered intravenously to patients, radioactivity found in urine was approximately 100-times that in plasma, suggesting that extracellular thymidine is rapidly excreted into urine. The elevated thymidine excretion reported here is therefore a potential marker of increased DNA breakdown and cell turnover due to γ radiation.

Elevated urinary excretion of N-hexanoylglycine is usually interpreted as a sign of impaired medium-chain fatty acid metabolism, that is, medium-chain acyl-CoA dehydrogenase (MCAD) deficiency, although this is usually accompanied by the excretion of dicarboxylic acids and free fatty acids. These additional metabolic signs did not appear in our metabolomic analysis, suggesting that the elevated appearance of N-hexanoylglycine in urine may not be a result of an effect of γ radiation on hepatic mitochondrial MCAD. N-hexanoylglycine urinary excretion is reduced 20-fold after activation of the nuclear receptor PPARα in mice and PPARα appears to play a role in the response of mice to 10 Gy γ irradiation.

Elevated taurine excretion in urine was first reported to be associated with carbon tetrachloride liver damage, but metabolomic studies have since characterized it as a general urinary marker of hepatotoxicity. As taurine is an end-product of cysteine catabolism, it has been proposed that urinary taurine excretion represents evidence of increased cysteine utilization in the liver, in response to toxic injury. The elevation in taurine urinary excretion reported here is modest and occurred only after the 8 Gy dose (FIG. 4E). Increased hepatic or renal cysteine/glutathione turnover is one possible explanation.

The elevation of urinary 3-hydroxy-2-methylbenzoic acid 3-O-sulfate after 3 Gy, but not 8 Gy, γ irradiation is without precedent of any kind. All possible isomers of this compound were synthesized in situ and evaluated by tandem mass spectrometry and this organic acid sulfate gave a perfect match to the urinary peak by both retention time and mass fragmentography (FIG. 3). To our knowledge, the parent 3-hydroxy-2-methylbenzoic acid has hitherto not been described in biological systems. Isomeric hydroxymethylbenzoic acids are, however, known bacterial metabolites and may arise from the gut flora.

In summary, a metabolomic investigation of γ radiation exposure in the mouse at 3 and 8 Gy doses is reported. OPLS analysis of mass spectrometric data matrices revealed novel biomarkers that were statistically significantly elevated in urine, and one biomarker that was statistically significantly attenuated in urine. GEDI self-organizing maps demonstrate the existence of dose-dependent excretion of a subset of global urinary biomarkers. These data will be useful to help design strategies for noninvasive radiation biodosimetry through metabolomics in human populations.

TABLE 1
Organic acids sulfated in situ to identify the urinary negative ion of 230.996 m/z

| | | retention time | principal ions of |
|---|---|---|---|
| | | of sulfate | sulfate [MS/MS] |
| isomer | structure | (min) | (abundance, %) |
| URINE PEAK | — | 2.12 | 230.995 (60), 187.004 (3), |
| | | | 151.038 (100), 107.050 (50) |
| 2-hydroxyphenyl- acetic acid | 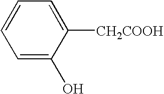 | 2.09 | 230.997 (100), 187.005 (70), 152.918 (30), 151.038 (20), 107.049 (80) |
| 3-hydroxyphenyl- acetic acid | 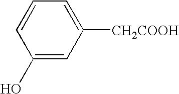 | 2.09 | 230.997 (100), 187.005 (90), 152.917 (40), 107.049 (20) |
| 4-hydroxyphenyl- acetic acid | 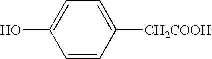 | 1.84 | 230.995 (100), 187.008 (30), 152.916 (20), 147.049 (0) |
| mandelic acid | 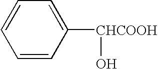 | 1.96 | 230.995 (25), 151.038 (35), 96.959 (100) |
| 3-methylsalicylic acid | 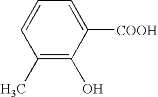 | 2.00 | 230.997 (25), 151.038 (35), 96.959 (100) |
| 4-methylsalicylic acid | 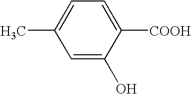 | 1.85 | 230.997 (15), 212.986 (100), 184.991 (25), 156.996 (10) |
| 5-methylsalicylic acid | 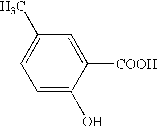 | 1.94 | 230.991 (45), 187.004 (15), 152.917 (100), 96.963 (75) |
| 6-methylsalicylic acid | 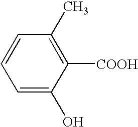 | 1.90 | 230.995 (100), 187.008 (30), 151.040 (5), 107.049 (50), 79.959 (40) |
| 3-hydroxy-2-methyl- benzoic acid | 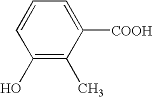 | 2.11 | 230.995 (50), 187.004 (3), 151.038 (100), 107.050 (70) |
| 3-hydroxy-4-methyl- benzoic acid | 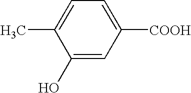 | 2.78 | 230.993 (40), 151.037 (100), 107.049 (65) |
| 3-hydroxy-5-methyl- benzoic acid | 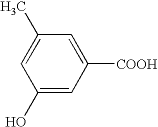 | 2.93 | 230.995 (100), 199.808 (35), 195.807 (70), 195.810 (60), 160.841 (50), 151.039 (10), 121.028 (40) |
| 5-hydroxy-2-methyl- benzoic acid | 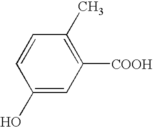 | 2.71 | 230.997 (60), 151.040 (100), 107.050 (90) |
| 4-hydroxy-2-methyl- benzoic acid | 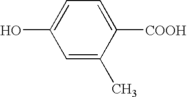 | 2.26 | 230.996 (100), 187.007 (40), 152.915 (15), 151.037 (10), 107.051 (45) |
| 4-hydroxy-3-methyl- benzoic acid | 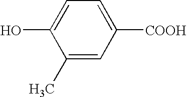 | 2.39 | 231.000 (100), 152.909 (60), 151.032 (15), 96.961 (40) |

TABLE 2
Identification of mouse urinary biomarkers after 3 Gy γ radiation

| RT | Mass | Mass | ppm | ion | |
|---|---|---|---|---|---|
| (min) | found | calc. | error | formula | identity |
| 2.29 | 230.9962 | 230.9963 | 1.3 | C8H7O6S⁻ | 3-Hydroxy- |
| | | | | | 2-methyl- |
| | | | | | benzoic |
| | | | | | acid O- |
| | | | | | sulfate |
| 3.65 | 172.0950 | 172.0974 | 13.9 | C8H14NO3⁻ | N-Hexanoyl- |
| | | | | | glycine |
| 1.90 | 241.0824 | 241.0824 | 0.0 | C10H13N2O5⁻ | β-Thymidine |
| 1.82 | 417.1143 | 417.1145 | 0.4 | C16H21N2O11⁻ | Putative |
| | | | | | thymidine |
| | | | | | 5′-β-D- |
| | | | | | glucuronide |
| 1.74 | 267.0741 | 267.0729 | 4.1 | C10H11N4O5⁻ | 2′-Deoxy- |
| | | | | | xanthosine |
| 0.65 | 151.0270 | 151.0256 | 9.3 | C5H3N4O2⁻ | Xanthine |
| 1.86 | 283.0700 | 283.0679 | 7.4 | C10H11N4O6⁻ | Xanthosine |
| 1.15 | 227.0660 | 227.0668 | 3.5 | C9H11N2O5⁻ | 2′-Deoxy- |
| | | | | | uridine |
| 0.54 | 228.1000 | 228.0984 | 7.0 | C9H12N3O4⁺ | 2′-Deoxy- |
| | | | | | cytidine |

TABLE 3
Identification of mouse urinary biomarkers after 8 Gy γ radiation

| RT | Mass | Mass | ppm | ion | |
|---|---|---|---|---|---|
| (min) | found | calc. | error | formula | identity |
| 3.66 | 172.0974 | 172.0974 | 0.0 | C8H14NO3⁻ | N-Hexanoyl- |
| | | | | | glycine |
| 0.29 | 124.0092 | 124.0068 | 24.2 | C2H6NO3S⁻ | Taurine |
| 1.90 | 241.0836 | 241.0824 | 3.7 | C10H13N2O5⁻ | β-Thymidine |
| 1.84 | 267.0806 | 267.0729 | 28.8 | C10H11N4O5⁻ | 2′-Deoxy- |
| | | | | | xanthosine |
| 1.80 | 283.0695 | 283.0679 | 5.6 | C10H11N4O6⁻ | Xanthosine |
| 1.15 | 227.0664 | 227.0668 | 1.8 | C9H11N2O5⁻ | 2′-Deoxyuridine |
| 0.60 | 228.0980 | 228.0984 | 1.8 | C9H12N3O4⁺ | 2′-Deoxycytidine |

TABLE 4
Historical biomarkers of radiation exposure

| | | Mono- | | | | |
|---|---|---|---|---|---|---|
| | Empirical | isotopic | | | | |
| Chemical Name | Formula | Mass (M) | MH+ | [M − H]− | Ref | Note |
| Glycine | C2H5NO2 | 75.032 | 76.0399 | 74.0242 | A | 1 |
| Thymine glycol | C5H8N2O4 | 160.0484 | 161.0562 | 159.0406 | B, C | 1 |
| Homovanillic acid | C9H10O4 | 182.0579 | 183.0657 | 181.0501 | D | 1 |
| 4-Hydroxy-3-methoxyphenylglycol | C9H12O4 | 184.0736 | 185.0814 | 183.0657 | D | 1 |
| Metanephrine | C10H15NO3 | 197.1052 | 198.113 | 196.0974 | D | 1 |
| 4-Hydroxy-3-methoxymandelic acid | C9H10O5 | 198.0528 | 199.0606 | 197.045 | D | 1 |
| Indoxyl sulfate | C8H7NO4S | 213.0096 | 214.0174 | 212.0018 | E | 1 |
| Thymidine glycol | C10H16N2O7 | 276.0958 | 277.1036 | 275.0879 | B | 1 |
| 8-Isoprostaglandin F_{2α} | C20H34O5 | 354.2406 | 355.2484 | 353.2328 | F, G | 1 |
| Thromboxane B₂ | C20H34O6 | 370.2355 | 371.2434 | 369.2277 | H | 1 |
| 8-Hydroxyguanine | C5H5N5O2 | 167.0443 | 168.0521 | 166.0365 | C | 2 |
| Creatinine | C4H7N3O | 113.0589 | 114.0667 | 112.0511 | I | 3 |
| Hippuric Acid | C9H9NO3 | 179.0582 | 180.0661 | 178.0504 | I | 3 |
| Normetanephrine | C9H13NO3 | 183.0895 | 184.0974 | 182.0817 | D | 3 |
| 5-Hydroxyindoleacetic acid | C10H9NO3 | 191.0582 | 192.0661 | 190.0504 | J-N | 3 |
| Citric Acid | C6H8O7 | 192.027 | 193.0348 | 191.0192 | A | 4 |

¹[M − H]− (±10 ppm) either not detected or not statistically significantly different (P ≧ 0.05) according to exposure status;
²[M − H]− (±10 ppm) elevated (P < 0.05) in urine from mice exposed to 3 Gy;
³[M − H]− (±10 ppm) elevated (P < 0.05) in urine from mice exposed to 8 Gy;
⁴[M − H]− (±10 ppm) elevated (P < 0.05) in urine from mice both exposed to 3 and 8 Gy. All comparisons made with mean creatinine-normalized relative concentrations by t test with α = 0.05.
A. V. E. Yushmanov, Evaluation of radiation injury by 1H and 31P NMR of human urine. Magn Reson Med 31, 48-52 (1994).
B. R. Cathcart, E. Schwiers, R. L. Saul and B. N. Ames, Thymine glycol and thymidine glycol in human and rat urine: a possible assay for oxidative DNA damage. Proc Natl Acad Sci USA 81, 5633-5637 (1984).
C. D. S. Bergtold, C. D. Berg and M. G. Simic, Urinary biomarkers in radiation therapy of cancer. Adv Exp Med Biol 264, 311-316 (1990).
D. D. Pericic, Z. Deanovic and S. Pavicic, Excretion of metabolites of biogenic amines in patients with irradiated brain tumours. Acta Radiol Ther Phys Biol 15, 81-90 (1976).
E. H. Smith and A. O. Langlands, Alterations in tryptophan metabolism in man after irradiation. Int J Radiat Biol Relat Stud Phys Chem Med 11, 487-494 (1966).
F. R. M. Wolfram, A. C. Budinsky, B. Palumbo, R. Palumbo and H. Sinzinger, Radioiodine therapy induces dose-dependent in vivo oxidation injury: evidence by increased isoprostane 8-epi-PGF(2 alpha). J Nucl Med 43, 1254-1258 (2002).
G. K. Camphausen, C. Menard, M. Sproull, F. Goley, S. Basu and C. N. Coleman, Isoprostane levels in the urine of patients with prostate cancer receiving radiotherapy are not elevated. Int J Radiat Oncol Biol Phys 58, 1536-1539 (2004).
H. M. J. Schneidkraut, P. A. Kot, P. W. Ramwell and J. C. Rose, Regional release of cyclooxygenase products after radiation exposure of the rat. J Appl Physiol 61, 1264-1269 (1986).
I. V. E. Yushmanov, Evaluation of radiation injury by 1H and 31P NMR of human urine. Magn Reson Med 31, 48-52 (1994).
J. Z. Deanovic, Z. Supek and M. Randic, Relationship Between The Dose Of Whole-Body X-Irradiation And The Urinary Excretion Of 5-Hydroxyindoleacetic Acid In Rats. Int J Radiat Biol Relat Stud Phys Chem Med 7, 1-9 (1963).
K. M. Randic and Z. Supek, Urinary excretion of 5-hydroxyindolacetic acid after a single whole-body x-irradiation in normal and adrenalectomized rats. Int J Radiat Biol 4, 151-153 (1961).
L. H. Smith and A. O. Langlands, Alterations in tryptophan metabolism in man after irradiation. Int J Radiat Biol Relat Stud Phys Chem Med 11, 487-494 (1966).
M. C. W. Scarantino, R. D. Ornitz, L. G. Hoffman and R. F. Anderson, Jr., On the mechanism of radiation-induced emesis: the role of serotonin. Int J Radiat Oncol Biol Phys 30, 825-830 (1994).
N. D. Pericic and Z. Deanovic, The metabolites of catecholamines in urine of patients irradiated therapeutically. Int J Radiat Biol Relat Stud Phys Chem Med 29, 367-376 (1976).

What is claimed:

1. A biomarker for determining gamma radiation exposure by an animal or human, comprising: 3-hydroxy-2-methylbenzoic acid 3-O-sulfate, N-hexanoylglycine, β-thymidine, taurine, xanthine, xanthosine, 2′-deoxyuridine, 2′-deoxycytidine, 2′-deoxyxanthosine, or any salt, ion, or combination thereof.

2. The biomarker of claim 1, wherein the biomarker determines the gamma radiation dose exposure by a human.

3. The biomarker of claim 1, wherein the biomarker comprises 3-hydroxy-2-methylbenzoic acid 3-O-sulfate, or any salt, ion, or combination thereof.

4. The biomarker of claim 1, wherein the biomarker comprises N-hexanoylglycine, or any salt, ion, or combination thereof.

5. The biomarker of claim 1, wherein the biomarker comprises β-thymidine, or any salt, ion, or combination thereof.

6. The biomarker of claim 1, wherein the biomarker comprises taurine, or any salt, ion, or combination thereof.

7. The biomarker of claim 1, wherein the biomarker comprises xanthine, or any salt, ion, or combination thereof.

8. The biomarker of claim 1, wherein the biomarker comprises xanthosine, or any salt, ion, or combination thereof.

9. The biomarker of claim 1, wherein the biomarker comprises 2′-deoxyuridine, or any salt, ion, or combination thereof.

10. The biomarker of claim 1, wherein the biomarker comprises 2′-deoxycytidine, or any salt, ion, or combination thereof.

11. The biomarker of claim 1, wherein the biomarker comprises 2′-deoxyxanthosine, or any salt, ion, or combination thereof.

12. The biomarker of claim 1, wherein the biomarker is characterized as being a specific metabolite in an animal or human exposed to a gamma radiation dose of at least about 3 Gy.

13. The biomarker of claim 1, wherein the biomarker is characterized as being a specific metabolite in an animal or human exposed to a gamma radiation dose of at least about 8 Gy.

14. The biomarker of claim 1, wherein the biomarker is characterized as being a specific metabolite in an animal or human exposed to a lethal gamma radiation dose.

15. The biomarker of claim 1, wherein the biomarker is characterized as being a specific metabolite in an animal or human exposed to a non-lethal gamma radiation dose.

16. A method for determining gamma radiation exposure by an animal or human, comprising:
(a) collecting a biological fluid from the animal or human;
(b) measuring the amount of one or more biomarkers specific to gamma radiation in the biological fluid; and
(c) correlating the amount of said biomarkers to the amount of gamma radiation exposure by the animal or human.

17. The method of claim 16, wherein the one or more biomarkers comprise a pyrimidine base, a purine base, a xanthine base, a pyrimidine nucleoside, a purine nucleoside, a xanthine nucleoside, a metabolite of a nucleic acid, a metabolite of fatty acid metabolism, or any salt, ion, or combination thereof.

18. The method of claim 17, wherein the one or more biomarkers comprise 3-hydroxy-2-methylbenzoic acid 3-O-sulfate, N-hexanoylglycine, β-thymidine, taurine, xanthine, xanthosine, 2′-deoxyuridine, 2′-deoxycytidine, 2′-deoxyxanthosine, or any ion, salt, or combination thereof.

19. The method of claim 16, wherein the amount of biomarkers is measured using chromatography, mass spectrometry, differential ion mobility spectroscopy, radioimmunoassay, nuclear magnetic resonance, infrared spectroscopy, visible spectroscopy, ultraviolet spectroscopy, immunological assay, calorimetric assay, Raman spectroscopy, capillary electrophoresis, or any combination thereof.

20. The method of claim 19, wherein ultra-performance liquid chromatography—time-of-flight mass spectrometry is used to determine the amount of the biomarkers.

21. The method of claim 16, wherein the biological fluid comprises, whole blood, blood plasma, blood serum, urine, breast milk, mucus, saliva, interstitial fluid, lymph, tears, sweat, sebum, semen, prostatic fluid, vaginal secretion, ear wax, or any combination thereof.

22. The method of claim 21, wherein the biological fluid comprises urine.

23. The method of claim 16, wherein the biological fluid is capable of being collected non-invasively.

24. The method of claim 16, wherein the amount of the biological biomarker is measured at a concentration in the range of from 1 pg/μl to 5000 pg/μl.

25. The method of claim 16, wherein the amount of the biological biomarker is measured at a concentration in the range of from 2 pg/μl to 2500 pg/μl.

26. The method of claim 16, wherein the amount of the biological biomarker is measured at a concentration in the range of from 5 pg/μl to 1000 pg/μl.

27. The method of claim 16, wherein steps (a)-(d) are carried out on a cancer patient who is undergoing, or has received, radiation treatment.

28. The method of claim 27, wherein steps (a)-(d) are carried out iteratively.

29. The method of claim 27, wherein the dose of radiation used to treat the cancer patient is controlled by the amount of one or more biomarkers specific to gamma radiation measured in the biological fluid.

30. The method of claim 16, wherein the step of (c) correlating the amount of said biomarkers to the amount of gamma radiation exposure by the animal or human involves the mathematical manipulation of the concentration of the one or more biomarkers.

31. The method of claim 30, wherein the mathematical manipulation comprises the functions of addition, subtraction, multiplication, and division.

32. The method of claim 31, wherein the mathematical manipulation of division is used to determine the ratio of the concentration of two or more of the biomarkers.

33. The method of claim 32, wherein the ratio of the concentration of the two of the biomarkers is in the range of from 1:10,000 to 10,000:1.

34. The method of claim 32, wherein the ratio of the concentration of the two of the biomarkers is in the range of from 1:100 to 100:1.

35. The method of claim 30, wherein the mathematical manipulation further comprises the manipulation of one or more constants.

36. A system for determining gamma radiation exposure by an animal or human, comprising:
a sample introduction section for collecting a fluid sample comprising one or more biomarkers for gamma radiation;
a volatilization section for volatilizing the fluid sample;
an ion source for ionizing a portion of the volatilized sample;
an ion mobility based filter for filtering out the at least one biomarker comprising an ion of a pyrimidine base, a purine base, a xanthine base, a pyrimidine nucleoside, a purine nucleoside, a xanthine nucleoside, a metabolite of a nucleic acid, a metabolite of fatty acid metabolism, or any combination thereof; and
a detector for detecting the at least one biomarker.

37. The system of claim 36, wherein the at least one biomarker comprises an ion of 3-hydroxy-2-methylbenzoic acid 3-O-sulfate, N-hexanoylglycine, β-thymidine, taurine, xanthine, xanthosine, 2′-deoxyuridine, 2′-deoxycytidine, or any combination thereof.

38. The system of claim 36, wherein the biomarker ions are capable of passing through the ion mobility filter in such a fashion that biomarker ions in the sample flows through an asymmetric field.

39. The system of claim 38, wherein the filter is configured to apply a compensation field to the asymmetric field to selectively pass ions through the filter.

40. The system of claim 39 comprising an electronic controller for controlling at least one condition of the filter.

41. The system of claim 40, wherein the controller is configured for storing information about filter conditions associated with filtering at least one known biomarker and adjusting the filter conditions to enable the at least one known biomarker to pass through the asymmetric field.

42. The system of claim 40, wherein the controller is configured for storing information about filter conditions associated with filtering a plurality of known markers and scanning the filter conditions to enable the plurality of known markers to pass through the asymmetric field.

43. The system of claim 36 comprising a gas chromatograph from which a portion of the sample is eluted before one of ionizing and pass through the ions.

44. The system of claim 36 comprising a pre-filter for filtering the sample using a membrane.

45. The system of claim 44, wherein the membrane comprises at least one polymer.

46. The system of claim 45, wherein the polymer comprises a perfluorinated polymer, a silicone, or any combination thereof.

47. The system of claim 36 comprising a pro-filter for removing unwanted components.

48. A method for treating an animal or human exposed to gamma radiation, comprising:
(a) collecting a biological fluid from the animal or human;
(b) measuring the amount of one or more biomarkers specific to gamma radiation in the biological fluid to develop a radiation exposure profile of the animal or human;
(c) correlating the radiation exposure profile to one or more compounds capable of counteracting the metabolic effects of the gamma radiation on the animal or human; and
(d) administering the one or more compounds to the animal or human.

49. The method of claim 48, wherein the radiation exposure profile correlates the one or more biomarkers to a change in the redox state of the cell.

50. The method of claim 49, wherein the change in the redox state of the cell arises from a generation of reactive oxygen species arising from the radiolysis of water molecules.

51. The method of claim 49, wherein the change in the redox state of the cell reduces enzymic reactions that are dependent upon one or more reduced nucleotide cofactors, flavin cofactors, or any combination thereof.

52. The method of claim 51, wherein the one or more reduced nucleotide cofactors or flavin cofactors comprise NADH, NADPH, FADH₂, or any combination thereof.

53. The method of claim 49, wherein the change in the redox state of the cell increases enzymic reactions that are dependent upon one or more oxidized nucleotide, flavin cofactors, or any combination thereof.

54. The method of claim 51, wherein the one or more oxidized nucleotide cofactors or flavin cofactors comprise NAD, NADP, FAD, or any combination thereof.

55. The method of claim 48, wherein a compound administered to the animal or human comprises N-acetylcysteine.

56. A biomarker for determining gamma radiation exposure by an animal or human, comprising a pyrimidine nucleoside, or any salt, ion, or combination thereof.

57. The biomarker of claim 56, wherein the pyrimidine nucleoside comprises a pyrimidine 2′-deoxyriboside, a pyrimidine riboside, or any salt, ion, or combination thereof.

58. The biomarker of claim 57, wherein the pyrimidine 2′-deoxyriboside comprises β-thymidine, 2′-deoxyuridine, 2′deoxycytidine, or any salt, ion, or combination thereof.

59. A biomarker for determining gamma radiation exposure by an animal or human, comprising a purine base, or any salt, ion, or combination thereof.

60. The biomarker of claim 59, wherein the purine base comprises xanthine, or any salt, ion, or combination thereof.

61. A biomarker for determining gamma radiation exposure by an animal or human, comprising a purine nucleoside, or any salt, ion, or combination thereof.

62. The biomarker of claim 61, wherein the purine nucleoside comprises a purine riboside, a purine deoxyriboside, or any salt, ion, or combination thereof.

63. The biomarker of claim 62, wherein the purine riboside comprises xanthosine, 2′-deoxyxanthosine, or any salt, ion, or combination thereof.

64. A biomarker for determining gamma radiation exposure by an animal or human, comprising a metabolite of a nucleic acid, or any salt, ion, or combination thereof.

65. The biomarker of claim 64, wherein the metabolite of a nucleic acid comprises 3-hydroxy-2-methylbenzoic acid 3-O-sulfate, N-hexanoylglycine, β-thymidine, taurine, xanthine, xanthosine, 2′-deoxyuridine, 2′-deoxycytidine, or any salt, ion, or combination thereof.

66. The biomarker of claim 64, wherein the metabolite of a nucleic acid is characterized as a metabolite of a flora contained within the gut of the animal or human.

67. The biomarker of claim 66, wherein the metabolite of the flora contained within the gut of the animal or human comprises 3-hydroxy-2-methylbenzoic acid 3-O-sulfate.

68. The biomarker of claim 64, wherein the metabolite of a nucleic acid is characterized as a marker for liver damage.

69. The biomarker of claim 68, wherein the marker for liver damage comprises taurine.

70. A biomarker for determining gamma radiation exposure by an animal or human, comprising a metabolite of fatty acid metabolism, or any salt, ion, or combination thereof.

71. The biomarker of claim 70, wherein the metabolite of fatty acid metabolism is found in the liver.

72. The biomarker of claim 70, wherein the metabolite of fatty acid metabolism comprises N-hexanoylglycine, an ion, salt, or any combination thereof.

73. A biomarker for determining gamma radiation exposure by an animal or human, comprising a xanthine base, ion, salt, or any combination thereof.

74. The biomarker of claim 73, wherein the biomarker comprises xanthosine, 2′-deoxyxanthosine, or any ion or salt, or any combination thereof.
